(19) United States
(12) Patent Application Publication
(10) Pub. No.: US 2021/0144975 A1
(43) Pub. Date: May 20, 2021

(54) AMERICAN COCKROACH PROCESSING EQUIPMENT FOR HOUSEHOLD FOOD WASTE

(71) Applicants: Guangmeiyuan R & D Center, Key Laboratory of Insect Developmental Biology and Applied Technology, So, Meizhou City (CN); Meizhou Huasheng Guangmei Biotechnology Co., Ltd., Meizhou City (CN)

(72) Inventors: Sheng LI, Meizhou City (CN); Na LI, Meizhou City (CN)

(73) Assignees: Guangmeiyuan R & D Center, Key Laboratory of Insect Developmental Biology and Applied Technology, So, Meizhou City (CN); Meizhou Huasheng Guangmei Biotechnology Co., Ltd., Meizhou City (CN)

(21) Appl. No.: 16/816,575

(22) Filed: Mar. 12, 2020

(30) Foreign Application Priority Data
Nov. 14, 2019 (CN) 201911110565.4

(51) Int. Cl. A01K 67/02; B03B 5/00

(52) U.S. Cl. CPC A01K 67/02; A01K 2227/706; B03B 5/00

(57) ABSTRACT

An American cockroach processing equipment for household food waste includes: a sorting apparatus, configured to perform solid-liquid separation of kitchen waste; a feeding and breeding apparatus, configured to receive solid waste separated by the sorting apparatus, and use the solid waste for cockroach breeding; and a waste liquid treatment apparatus, configured to receive waste liquid separated by the sorting apparatus, and preferably configured to use the waste liquid for detergent production. The equipment can first perform solid-liquid separation of kitchen waste, and then use the separated solid waste to breed the cockroaches that are used to produce high-grade protein feeds and pharmaceutical raw materials, therefore better economic benefits are achieved and the waste is effectively reused.

### FIELD

The present disclosure relates to an American cockroach processing equipment for household food waste, belonging to the field of biological treatment technology of kitchen waste.

### BACKGROUND

Kitchen waste is living waste produced by residents in the living consumption process, and its main ingredients include rice and flour food residue, vegetable, oil and grease, meat and bone. Kitchen waste includes chemical constituents such as amylum, cellulose, protein, lipid, and inorganic salt. Current treatment of kitchen waste is not reasonable because it needs to be collected and transported by using numerous manpower and material resources, causing high costs, and kitchen waste is produced in a large amount every day, which is difficult to deal with, burning and filling seriously polluting the environment, particularly under the current environment of environmental protection, waste reuse, and resource utilization. It is urgent to find a safe and pollution-free biological treatment solution.

### SUMMARY

The present disclosure is intended to resolve at least one of the technical problems in the prior art. For this reason, the present disclosure provides an American cockroach processing equipment for household food waste. American cockroaches can be produced as high-grade protein feeds and pharmaceutical raw materials, and the equipment may be directly disposed near the canteen and hotel, which is the source of a large amount of kitchen waste to reduce time and costs for transportation and collection. The equipment includes:

a sorting apparatus, configured to perform solid-liquid separation of kitchen waste;

a feeding and breeding apparatus, configured to receive solid waste separated by the sorting apparatus, and use the solid waste for cockroach breeding; and

a waste liquid treatment apparatus, configured to receive waste liquid separated by the sorting apparatus.

Further, the waste liquid treatment apparatus is configured to use the waste liquid for detergent production.

According to an embodiment of the present disclosure, at least the following technical effects are obtained:

The equipment can first perform solid-liquid separation of kitchen waste, and then use the separated solid waste for cockroach breeding. Because cockroaches can be used to produce high-grade protein feeds and pharmaceutical raw materials, better economic benefits are achieved and waste is effectively reused. In addition, the equipment can be directly placed at the source, so that a user only needs to deliver produced kitchen waste to the equipment. This greatly reduces time costs and transportation costs and provides high practicability in comparison with a conventional means.

According to some embodiments of the present disclosure, the feeding and breeding apparatus includes:

a cockroach breeding tank, provided with a plurality of cockroach breeding units;

a feeding means, disposed between the sorting apparatus and the cockroach breeding tank, and configured to deliver solid waste separated by the sorting apparatus to the cockroach breeding tank; and

an escape prevention means, disposed in at least one of the cockroach breeding tank, the feeding means, and the sorting apparatus, and configured to prevent cockroaches from escaping.

According to some embodiments of the present disclosure, the feeding means are provided a plurality of conveyor belts up and down in the cockroach breeding tank, and the feeding means is provided with a feeding pipe corresponding to each of the conveyor belts, the feeding pipe is provided with a feeder, the plurality of the cockroach breeding units are arranged at a plurality of layers corresponding to the conveyor belts, the cockroach breeding units at each layer are distributed on both sides of the conveyor belts, and an extension edge is provided between the cockroach breeding unit and the conveyor belt to reduce or eliminate a gap between the two.

According to some embodiments of the present disclosure, a first scraper is disposed at the bottom of the conveyor belt, and the first scraper can abut against the conveyor belt to clean residue remaining on the conveyor belt.

According to some embodiments of the present disclosure, the bottom of the cockroach breeding tank is provided with a cockroach platform that may be opened through downward rotation, a discharge means is disposed below the cockroach platform, the cockroach breeding tank is provided with a second scraper above the cockroach platform, and the second scraper is configured to clean the residue at the bottom of the cockroach breeding tank.

According to some embodiments of the present disclosure, an output end of the discharge means is connected to a microwave inactivation apparatus.

According to some embodiments of the present disclosure, the microwave inactivation apparatus is provided with an input movable door and an output movable door, the input movable door corresponds to the output end of the discharge means, and only one of the input movable door and the output movable door is opened at a time in an operating state.

According to some embodiments of the present disclosure, the cockroach breeding tank has an inner support structure and an outer housing structure, the cockroach breeding unit is disposed on the inner support structure, the cockroach breeding tank is provided with a movable observation door that may be opened or closed, corresponding to the cockroach breeding unit, and the cockroach breeding tank is provided with a zipper plastic mesh on an inner side of the movable observation door.

According to some embodiments of the present disclosure, the cockroach breeding tank is connected to an air purification system, and a filter and sound insulation cotton are disposed at a connecting joint between the air purification system and the cockroach breeding tank.

According to some embodiments of the present disclosure, a chassis body is further included, and the cockroach breeding tank, the sorting apparatus, and the waste liquid treatment apparatus are disposed in parallel in the chassis body, where the sorting apparatus and the waste liquid treatment apparatus are arranged in parallel at one end of the cockroach breeding tank, the chassis body is provided with an automatic feeding machine on a sidewall close to the sorting apparatus, the sorting apparatus includes a flushing means, a feeder, a pulverizer, and a solid-liquid separator that are arranged from top to bottom, the flushing means is configured to flush the feeder, the pulverizer is configured to crush kitchen waste delivered by the feeder, and the solid-liquid separator is configured to perform solid-liquid separation of the crushed kitchen waste.

Some of additional aspects and advantages of the present disclosure are provided in the following description, and some become apparent from the following description, or are appreciated by the practice of the present disclosure.

### BRIEF DESCRIPTION OF THE DRAWINGS

The following further describes the present disclosure with reference to accompanying drawings and embodiments.

FIG. 1 is a schematic structural diagram of a first view angle of the disclosure; and

FIG. 2 is a schematic structural diagram of a second view angle of the disclosure.

### DETAILED DESCRIPTION

The following describes in detail embodiments of the present disclosure. Examples of the embodiments are shown in the accompanying drawings, where identical or similar reference numerals represent identical or similar elements or elements having identical or similar functions. The embodiments described below with reference to the accompanying drawings are exemplary and are intended only to explain the present disclosure and not to be construed as limiting the present disclosure.

In the description of the present disclosure, it should be understood that the azimuth or positional relationship related to the description of the azimuth, for example, the azimuth or positional relationship indicated by the upper, lower, front, rear, left, and right, is based on the azimuth or positional relationship shown in the accompanying drawings and is intended only to facilitate the description and simplification of the present disclosure, and is not intended to indicate or imply that the indicated apparatus or element must have a specific azimuth and be constructed and operated in a specific azimuth and therefore cannot be construed as limiting the present disclosure.

In the description of the present disclosure, “several” means one or more; “a plurality of” means more than two; “greater than”, “less than”, “exceed” and the like are understood as excluding the given number; “more than”, “below”, “within”, and the like are understood as including the given number. “First” and “second” (if described) are merely used to distinguish between technical features and are not understood as indicating or implying relative importance or implicitly indicating a quantity of indicated technical features or implicitly indicating a sequential relationship of the indicated technical features.

In the description of the present disclosure, unless otherwise expressly limited, words such as disposing, installation, and connection shall be understood broadly. A person skilled in the art may reasonably determine the specific meaning of the foregoing words in the present disclosure with reference to specific content of the technical solution.

An embodiment of the present disclosure provides an American cockroach processing equipment for household food waste, including:

a sorting apparatus, configured to perform solid-liquid separation of kitchen waste;

a feeding and breeding apparatus, configured to receive solid waste separated by the sorting apparatus, and use the solid waste for cockroach breeding; and

a waste liquid treatment apparatus, configured to receive waste liquid separated by the sorting apparatus, and preferably configured to use the waste liquid for detergent production.

The equipment can first perform solid-liquid separation of kitchen waste, and then use the separated solid waste for cockroach breeding. Because cockroaches can be used to produce high-grade protein feeds and pharmaceutical raw materials, better economic benefits are achieved and waste is effectively reused.

In addition, the equipment can be directly placed at the breeding source, so that a user only needs to deliver produced kitchen waste to the equipment. This greatly reduces time costs and transportation costs and provides high practicability in comparison with a conventional means.

Compared with some kitchen waste treatment devices on the market, the kitchen waste equipment in the present disclosure can further treat the separated waste liquid on the basis of solid-liquid separation treatment, and make it into detergent which can be directly used or sold, bringing better economic benefit and treatment effect than similar devices on the market, and gaining strong market competitiveness.

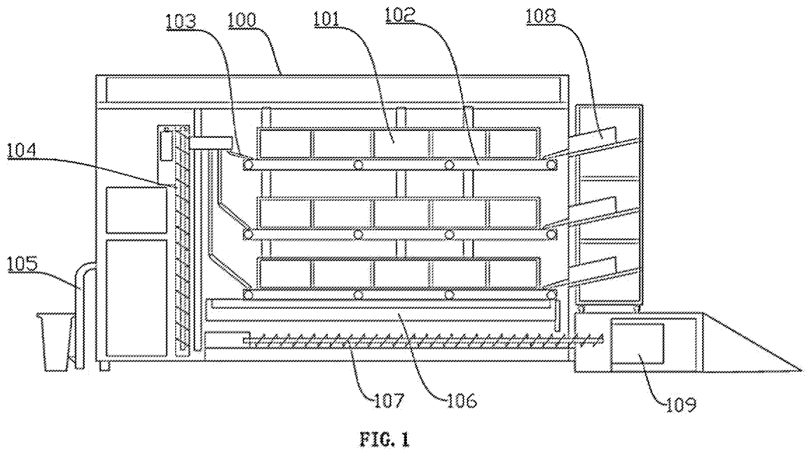

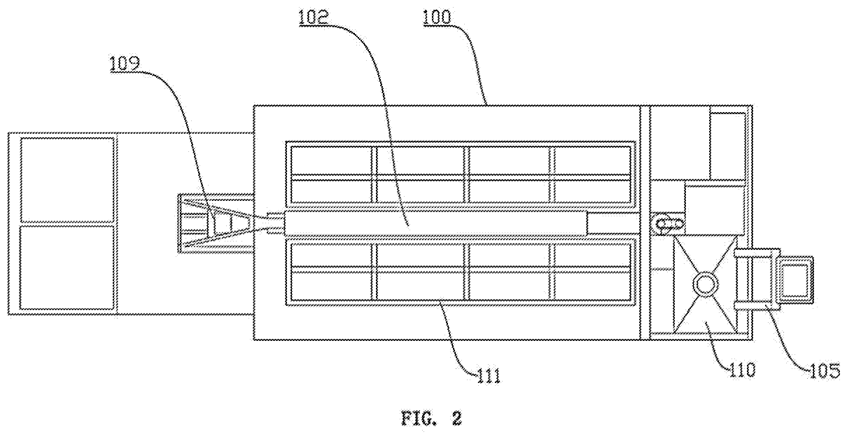

Referring to FIG. 1 and FIG. 2, in some embodiments of the present disclosure, the equipment is provided with a chassis body 100, and the sorting apparatus, the feeding and breeding apparatus, and the waste liquid treatment apparatus are all disposed in the chassis body 100, so as to maintain integrity of the equipment and facilitate placement and transportation of the equipment. In addition, an internal structure can be protected by the chassis body 100, thereby extending a service life.

In some embodiments of the present disclosure, the feeding and breeding apparatus includes:

a cockroach breeding tank, provided with a plurality of cockroach breeding units 101;

a feeding means, disposed between the sorting apparatus and the cockroach breeding tank, and configured to deliver solid waste separated by the sorting apparatus to the cockroach breeding tank; and

an escape prevention means, disposed in at least one of the cockroach breeding tank, the feeding means, and the sorting apparatus, and configured to prevent cockroaches from escaping.

Preferably, the feeding means are provided a plurality of conveyor belts 102 up and down in the cockroach breeding tank, the feeding means is provided with a feeding pipe 103 corresponding to each of the conveyor belts 102, and the feeding pipe 103 is provided with a feeder. In this way, each feeding pipe 103 can be fed accurately by a feeder, so that each conveyor belt 102 can be fed quantitatively. To prevent cockroaches from entry, the feeding pipe 103 is provided with a solenoid valve at one end close to the conveyor belt 102.

Correspondingly, preferably, the plurality of the cockroach breeding units 101 are arranged at a plurality of layers corresponding to the conveyor belts 102, and the cockroach breeding units 101 at each layer are distributed on both sides of the conveyor belts 102. The cockroach breeding unit 101 is of a non-closed structure, and has, on its sidewalls, several gaps that can be passed by cockroaches. The cockroach breeding units 101 are disposed on both sides of the conveyor belt 102, so that the cockroaches can independently find food and crawl from the cockroach breeding unit 101 to the conveyor belt 102 for taking food. The multi-layer arrangement can improve a breeding capacity. Apparently, in an actual production process, single-layer arrangement may also be used. In addition, the cockroach breeding units 101 may be disposed on only one side of the conveyor belt 102 according to an actual quantity or demand. Alternatively, the conveyor belt 102 is removed, and the kitchen waste is directly delivered to the cockroach breeding unit 101 through the feeding pipe 103.

The structures such as the feeder and the solenoid valve belong to the common knowledge or are products sold on the market, and in a production process, the common knowledge may be referenced or such accessories may be purchased for use. Therefore, specific structures are not described in detail herein.

To prevent cockroaches from falling while crawling from the cockroach breeding unit 101 to the conveyor belt 102, or to prevent a case in which cockroaches cannot return smoothly from the conveyor belt 102 to the cockroach breeding unit 101, preferably, an extension edge is provided between the cockroach breeding unit 101 and the conveyor belt 102 to reduce or eliminate a gap between the two.

Generally, the conveyor belt 102 is of a closed structure, and therefore may be divided into an upper transport section and a lower return section, and the foregoing extension edge may be disposed between the upper transport section and the lower return section. Certainly, another structure that enables cockroaches to crawl back to the cockroach breeding unit 101 after falling may also be disposed. Such a structure can also prevent cockroaches from escaping to a certain extent, so that the cockroaches stably remain at each layer of cockroach breeding units 101.

In some embodiments, the cockroach breeding unit 101 is provided with a similar structure on the top and at the bottom in addition to a gap on the sidewall that can be passed by the cockroach, so that cockroach feces or dead bodies or the like at an upper layer can fall off. Correspondingly, in some embodiments, the bottom of the cockroach breeding tank is provided with a cockroach platform that may be opened through downward rotation, a discharge means 107 is disposed below the cockroach platform, the cockroach breeding tank is provided with a second scraper above the cockroach platform, and the second scraper is configured to clean the residue at the bottom of the cockroach breeding tank.

The second scraper and the cockroach platform are disposed in many ways, where several examples are described below.

Example 1: An elongated hopper 106 is disposed at the bottom of the cockroach breeding tank. The hopper 106 is located below the conveyor belt 102 and maintains a length consistent with a conveying direction of the conveyor belt 102. The discharge means 107 is located below the hopper 106, and the cockroach platform is rotatably or translationally disposed within the hopper 106 to close the hopper 106 in a normal state. Two second scrapers are respectively disposed at the bottoms of the cockroach breeding tanks on both sides of the hopper 106. During operation, the two second scrapers move to the middle, to push the residue or cockroach bodies at the bottom of the cockroach breeding tank to the position of the hopper 106, and then the cockroach platform is opened under control for discharging. Herein, the opening of the cockroach platform may be separately driven, or may be implemented under the action of the second scraper.

Example 2: The second scraper is installed on the cockroach platform. When the residue is cleaned, the cockroach platform is opened downwards and then the second scraper is controlled to move from a pivotal end of the cockroach platform to the open end, so that the residue on the upper end surface of the cockroach platform is cleaned through scrapping.

Example 3: The second scraper is installed on the cockroach breeding tank, and its moving path is tilted. The cockroach platform is provided with a reset structure that keeps the cockroach platform horizontal in normal state. During the movement, a lower end of the second scraper abuts against the cockroach platform, and pushes the cockroach platform away.

Certainly, in an actual production process, there are further a plurality of other implementations for their cooperative movement, without detail description herein.

Arrangement of the cockroach platform can effectively avoid discharging falling live cockroaches, which results in waste, and allow live cockroaches to crawl back again. Combined with the effect of the second scraper, it is possible to avoid discharging live cockroaches that crowd above the discharge means 107, thereby improving a sealing property of the equipment.

In some embodiments, a first scraper is disposed at the bottom of the conveyor belt 102, that is, a first scraper is disposed at the lower return section of the conveyor belt 102, and the first scraper can abut against a surface of the lower return section of the conveyor belt 102 to clean the residue remaining on the conveyor belt 102. In the actual production process, the first scraper may always abut against the conveyor belt 102, or the first scraper may be movably disposed so that the first scraper abuts against the conveyor belt 102 only when needed for cleaning.

In some embodiments, the discharge means 107 is provided as a screw push-out means that pushes out residue or the like through screw rotation. Because such residue may contain live cockroaches, and include many residues, for the sake of environmental protection, preferably, an output end of the discharge means 107 is connected to a microwave inactivation apparatus 109. First, microwave inactivation treatment is performed on the discharged residue by the microwave inactivation apparatus 109, and then the inactivated residue is taken out for treatment.

To facilitate taking-out of the inactivated residue, in some embodiments, the microwave inactivation apparatus 109 is provided with an input movable door and an output movable door, the input movable door corresponds to the output end of the discharge means 107, and only one of the input movable door and the output movable door is opened at a time in an operating state. In this way, when the discharge means 107 performs discharging, the input movable door is opened to facilitate the introduction of residue, and then the input movable door is closed for inactivation processing. After the inactivation processing is completed, the output movable door is opened to remove the residue.

The microwave inactivation apparatus 109 disposed in this way can effectively prevent cockroaches from escaping, and is convenient for users to operate, the introduction and removal of residue do not conflict.

Considering that the discharge means 107 does not have a function of completely feeding the residue into the microwave inactivation apparatus 109, a downwardly inclined discharge channel may be disposed at the output end of the discharge means 107, and the end of the discharge channel corresponds to the input movable door, so that when the input movable door is opened, the residue can be smoothly fed into the microwave inactivation apparatus 109 along the discharge channel under the action of gravity and inertia. A structure of the microwave inactivation apparatus 109 may be arranged with reference to a structure of a microwave oven. Therefore, the specific structure of the microwave inactivation apparatus 109 is not described in detail herein.

In some embodiments, because the equipment is kept closed by using the chassis body 100, to ensure that cockroaches are in a good breeding environment, the cockroach breeding tank is preferably connected to an air purification system, which ensures internal air quality. Apparently, there is a corresponding pipeline connection between the air purification system and the cockroach breeding tank. To prevent cockroaches from escaping by entering the air purification system through the pipeline or affecting the operation of the air purification system, a filter and sound insulation cotton are disposed at a connecting joint between the air purification system and the cockroach breeding tank.

In practice, a temperature of air inside the cockroach breeding tank is preferably controlled at 20° C.-28° C., preferably 25° C., and humidity is controlled at 60%-90%, preferably 65%. Such control may be implemented by evenly distributing multi-point temperature and humidity sensors in the cockroach breeding tank for collecting data, and then obtaining an optimal value through analysis by software of an electric appliance control part. The optimal temperature and humidity are controlled by constituent means of the air purification system such as a temperature controller and a dehumidifier. When abnormal temperature and humidity signals are detected, an alarm function may be set to notify manual processing, and when necessary, an automatic shutdown function may be set.

In addition, a camera may be disposed inside the cockroach breeding tank to monitor a real-time status of cockroaches, and a display screen may be disposed on a surface of the chassis body 100 to display a camera picture, or an operation panel of another electrical structure.

To facilitate an operation performed inside the chassis body 100, an LED indicator may be disposed inside the chassis body 100.

In some embodiments of the present disclosure, the cockroach breeding tank has an inner support structure 111 and an outer housing structure, the cockroach breeding unit 101 is disposed on the inner support structure 111, the cockroach breeding tank is provided with a movable observation door that may be opened or closed, corresponding to the cockroach breeding unit 101, and the cockroach breeding tank is provided with a zipper plastic mesh on an inner side of the movable observation door.

In this way, placement and installation of a plurality of layers of or a plurality of cockroach breeding units 101 can be implemented by the arrangement of the inner support structure 111, and the outer housing structure can effectively ensure appearance of the equipment. In addition, arrangement of the zipper plastic mesh can also prevent cockroaches from escaping.

With reference to the foregoing content, it can be learned that in some embodiments, the cockroach breeding tank has a plurality of electrical structures, that is, internally has a circuit and a wire structure. For convenience of management and maintenance, the cockroach breeding tank is preferably divided into three layers: an upper layer, a middle layer, and a lower layer. All cockroach breeding units 101 are distributed at the middle layer, the upper layer is used to arrange pipelines, and the lower layer includes the discharge means 107 and corresponding support structures such as a foot cup and movable support wheels. In addition, door structures are disposed in different corresponding areas on sidewalls of the cockroach breeding tank to facilitate corresponding operations on the internal structure.

The cockroach breeding tank arranged in this way separates modules and components of different functions, so that a cockroach breeding area is not affected during maintenance and repair, and cockroaches are prevented from escaping along a pipeline path.

To properly utilize internal space of the chassis body 100 and implement reasonable layout, in some embodiments, the cockroach breeding tank, the sorting apparatus, and the waste liquid treatment apparatus are disposed in parallel in the chassis body 100, where the sorting apparatus and the waste liquid treatment apparatus are arranged in parallel at one end of the cockroach breeding tank, the chassis body 100 is provided with an automatic feeding machine 105 on a sidewall close to the sorting apparatus, the sorting apparatus includes a flushing means, a feeder 110, a pulverizer, and a solid-liquid separator that are arranged from top to bottom, the flushing means is configured to flush the feeder 110, the pulverizer is configured to crush kitchen waste delivered by the feeder 110, and the solid-liquid separator is configured to perform solid-liquid separation of the crushed kitchen waste.

A structure of the automatic feeding machine 105 is the same as a structure on the market that can be used to lift and/or slip a rubbish bin, for example, a lifting means on a garbage truck. Details are not described herein.

Because the solid-liquid separator is located at the bottom, the feeding means is also provided with a conveying means such as a chain conveyor and a peristaltic pump for lifting solid waste. Such a means is disclosed in a well-known technology, and therefore no further description is provided herein.

During operation, kitchen waste is directly dumped into the feeder 110, or dumped into the feeder 110 via the automatic feeding machine 105. The pulverizer crushes the kitchen waste, performs solid-liquid separation after the kitchen waste is crushed to a specific particle size, and delivers solid waste to the cockroach breeding tank and delivers waste liquid to the waste liquid treatment apparatus. In this process, a sequence of solid-liquid separation and crushing may be changed. The flushing means is provided with an ejector that acts on a sidewall of the feeder 110, to clean residual kitchen waste thereon. For operation safety consideration, a cover plate is disposed on the feeder 110. The cover plate is opened before kitchen waste is dumped, and closed after the kitchen waste is dumped.

As shown in the figure, in some embodiments, a cockroach adding means 108 is disposed at an end of the chassis body 100 away from the sorting apparatus and the waste liquid treatment apparatus. Because the chassis body 100 is provided with the automatic feeding machine 105 at an end close to the sorting apparatus and the waste liquid treatment apparatus, if the cockroach adding means 108 is disposed at this place, interference easily occurs and it is difficult to implement. However, on two sides of the chassis body 100 that are adjacent to the sorting apparatus and the waste liquid treatment apparatus, it is difficult to implement average delivery to the plurality of cockroach breeding units 101 at each layer. Only at the end of the chassis body 100 away from the sorting apparatus and the waste liquid treatment apparatus, the bottom of this end is provided with a microwave inactivation apparatus 109, but there is mounting space above it, and this end enables the cockroach adding means 108 to directly connect to the conveyor belt 102, so that cockroaches can enter each cockroach breeding unit 101 at each layer through the conveyor belt 102. In addition, because food is available on the conveyor belt 102, adding is facilitated. Therefore, arrangement at this position is an optimal solution.

Layer distribution of the cockroach adding means 108 is in a one-to-one correspondence with layer distribution of the cockroach breeding unit 101. Specifically, there is an adding movable window on the sidewall of the chassis body 100, and an inclined groove is arranged on the inner wall of the chassis body 100 corresponding to the adding movable window. Cockroaches may be added by opening the adding movable window. A sealing strip is provided between the adding movable window and the sidewall of the chassis body 100 to prevent cockroaches from escaping.

Apparently, in the above-mentioned structures, either the arrangement of the extension edge or the arrangement of structures such as the zipper plastic mesh and the cover plate is one of escape prevention means.

In some embodiments, the waste liquid treatment apparatus is provided with an oil-water separation means, and waste oil is first separated from the waste liquid by the oil-water separation means, and waste water is discharged to a designated pipe network for further treatment. Then the waste oil is treated. For example, a treatment process of a conventional gutter oil chemical treatment method is as follows: crude oil deslagging→crude oil dehydration (adding dehydrant)→crude oil sterilization (high temperature)→crude oil decolorization (adding decolorant)→crude oil deodorization (adding deodorant)→refining, stabilization, and adjustment (adding other additives, removing other foreign matters, and adjusting and stabilizing final ingredients)→saponification (adding alkaline substance)→finished goods production.

Because the equipment structure, process setting, and the like for such reaction treatment belong to the common knowledge, no further description is provided herein.

In view of a large quantity and variety of equipments, a large footprint, high costs, high operation costs, high output costs, and lack of economic benefit of this conventional treatment technology, the present disclosure provides a new treatment technology. The process route of the new treatment technology is as follows: adding species of bacteria to crude oil→bio-chemical reaction of some species of bacteria and residue→bio-chemical reaction of some species of bacterials→bio-chemical reaction of some species of bacteria and pigment substance→bio-chemical reaction of some species of bacteria and grease→saponification of alkaline substance→deodorization (ozone deodorization)→heating and dehydration of bio-chemical reaction→finished goods production.

The specific reaction process is as follows:

1. Gutter oil and mixed auxiliary materials are put into the feed port, and the machine starts to work. Stirring, heating, gas exhaustion, sterilization, deodorization, and air purification are started.

2. Bio-chemical reaction is carried out on some species of bacteria and residues in the waste grease. Through aerobic reaction, the residues are produced as friction auxiliary agents for promoting detergency.

3. Bio-chemical reaction is carried out on some species of bacteria and residual bacterial substance in the waste grease. Through aerobic reaction and inactivation, auxiliary agents for promoting detergency are produced.

4. Bio-chemical reaction is carried out on some species of bacterials in the waste grease. Through aerobic reaction, other auxiliary agents are produced.

5. Through aerobic reaction, some species of bacteria and the grease whose ingredients are adjusted are produced as lipid substance having a shorter molecular chain.

6. Saponification reaction is carried out on alkaline substance and lipid substance having a shorter molecular chain.

7. An ozone generator performs sterilization and deodorization.

8. The heat produced by the reaction is dehydrated and dried together with other heat sources.

9. The resultant mixed detergent substance is pulverized and discharged.

A plurality of apparatuses using this reaction process may be integrated into one reactor. For example, a high temperature heating apparatus, a crushing and stirring integration apparatus, an automatic discharge apparatus, an ozone apparatus, a dust interception apparatus, and a gas exhaustion apparatus are integrated to form a reactor capable of completing the foregoing entire reaction process. Because constitution of such apparatuses can be learned from the common knowledge, no further description is provided herein.

The proportion and effect of the mixed auxiliary materials added in the above-mentioned process step 1 are as follows:

1. alkaline substance, 5%-50%, saponification of grease;

2. organic acid, 2%-15%, adjusting a PH value;

3. aerobic bacteria group, 0.1%-15%, aerobic reaction, degradation of organic macromolecules;

4. protease, 0.1%-5%, promoting reaction and detergency;

5. glucose, 5%-15%, promoting reaction and preserving strains;

6. sodium alginate, 1%-5%, preserving strains;

7. other auxiliary agents, 1%-10%, promoting detergency.

The foregoing reactor is used to perform reaction processing, and after a reaction cycle (5-60 minutes), the waste oil can be produced as powdery detergents, which are then discharged through a discharge apparatus and automatically discharged into a vessel.

These powdery detergents, used in combination with water, can wash various commonly seen dirts and oil stains. The powdery detergents have advantages of compliance with the national inspection standards, strong decontamination, no injury to hands, and low costs.

This technology is also applicable to other biological oils to produce detergents on site. The produced detergents have advantages of strong decontamination, no injury to hands, compliance with stricter standards, and higher level of safety. In addition, the produced detergent powder can be directly used or can be sold, yielding remarkable economic benefits.

The specific parameters of the above-mentioned process may be set by referring to the Chinese Patent Application No. CN201410844811.X. Details are not described herein.

In conclusion, according to the kitchen waste equipment in the present disclosure, solid-liquid separation is performed on kitchen waste, solid waste is used to breed cockroaches, and waste liquid is used to produce detergents. Cockroaches can be produced as high-grade protein feeds and pharmaceutical raw materials and detergents can be directly used. Therefore, the equipment has excellent economic benefits and practicability. Furthermore, the equipment may be directly disposed at the source to reduce time and costs for transportation and collection, and is suitable for a place at which a waste treatment capacity at the source is relatively small and there is no large-scale treatment space.

In the description of this specification, reference to the terms “one embodiment”, “some embodiments”, “exemplary embodiments”, “examples”, “specific examples”, “some examples”, and the like means that specific features, structures, materials, or characteristics described in connection with the embodiment or example are included in at least one embodiment or example of the present disclosure. In this specification, exemplary expressions of the foregoing terms do not necessarily refer to the same embodiments or examples. Moreover, the described specific features, structures, materials, or characteristics may be combined in an appropriate manner in any one or more embodiments or examples.

Although the embodiments of the present disclosure have been shown and described, a person of ordinary skill in the art may appreciate that various changes, modifications, replacements, and variations may be made to these embodiments without departing from the principles and purposes of the present disclosure, and the scope of the present disclosure shall be subject to the claims and equivalents thereof.

1. An American cockroach processing equipment for household food waste, comprising:
a sorting apparatus, configured to perform solid-liquid separation of kitchen waste;
a feeding and breeding apparatus, configured to receive solid waste separated by the sorting apparatus, and use the solid waste for cockroach breeding; and
a waste liquid treatment apparatus, configured to receive waste liquid separated by the sorting apparatus.

2. The equipment according to claim 1, wherein the feeding and breeding apparatus comprises:
a cockroach breeding tank, provided with a plurality of cockroach breeding units;
a feeding means, disposed between the sorting apparatus and the cockroach breeding tank, and configured to deliver solid waste separated by the sorting apparatus to the cockroach breeding tank; and
an escape prevention means, disposed in at least one of the cockroach breeding tank, the feeding means, and the sorting apparatus, and configured to prevent cockroaches from escaping.

3. The equipment according to claim 2, wherein the feeding means are provided a plurality of conveyor belts up and down in the cockroach breeding tank, and the feeding means is provided with a feeding pipe corresponding to each of the conveyor belts, the feeding pipe is provided with a feeder, the plurality of the cockroach breeding units are arranged at a plurality of layers corresponding to the conveyor belts, the cockroach breeding units at each layer are distributed on both sides of the conveyor belts, and an extension edge is provided between the cockroach breeding unit and the conveyor belt to reduce or eliminate a gap between the two.

4. The equipment according to claim 3, wherein a first scraper is disposed at the bottom of the conveyor belt, and the first scraper can abut against the conveyor belt to clean residue remaining on the conveyor belt.

5. The equipment according to claim 3, wherein the bottom of the cockroach culture tank is provided with a cockroach platform that may be opened through downward rotation, a discharge means is disposed below the cockroach platform, the cockroach breeding tank is provided with a second scraper above the cockroach platform, and the second scraper is configured to clean the residue at the bottom of the cockroach breeding tank.

6. The equipment according to claim 5, wherein an output end of the discharge means is connected to a microwave inactivation apparatus.

7. The equipment according to claim 6, wherein the microwave inactivation apparatus is provided with an input movable door and an output movable door, the input movable door corresponds to the output end of the discharge means, and only one of the input movable door and the output movable door is opened at a time in an operating state.

8. The equipment according to claim 2, wherein the cockroach breeding tank has an inner support structure and an outer housing structure, the cockroach breeding unit is disposed on the inner support structure, the cockroach breeding tank is provided with a movable observation door that may be opened or closed, corresponding to the cockroach breeding unit, and the cockroach breeding tank is provided with a zipper plastic mesh on an inner side of the movable observation door.

9. The equipment according to claim 2, wherein the cockroach breeding tank is connected to an air purification system, and a filter and sound insulation cotton are disposed at a connecting joint between the air purification system and the cockroach breeding tank.

10. The equipment according to claim 2, further comprising a chassis body, wherein the cockroach breeding tank, the sorting apparatus, and the waste liquid treatment apparatus are disposed in parallel in the chassis body; the sorting apparatus and the waste liquid treatment apparatus are arranged in parallel at one end of the cockroach breeding tank; the chassis body is provided with an automatic feeding machine on a sidewall close to the sorting apparatus; the sorting apparatus comprises a flushing means, a feeder, a pulverizer, and a solid-liquid separator that are arranged from top to bottom; the flushing means is configured to flush the feeder; the pulverizer is configured to crush kitchen waste delivered by the feeder; and the solid-liquid separator is configured to perform solid-liquid separation of the crushed kitchen waste.
